(19) United States
(12) Patent Application Publication
(10) Pub. No.: US 2019/0167409 A1
(43) Pub. Date: Jun. 6, 2019

(54) DEVICES AND METHOD FOR MODIFYING BLOOD PRESSURE IN THE LUNGS AND PULMONARY VASCULATURE BY IMPLANTING FLOW MODIFIER(S) IN PULMONARY VEIN(S)

(71) Applicant: 4C Medical Technologies, Inc., Brooklyn Park, MN (US)

(72) Inventors: Philippe Genereux, New York, NY (US); Robert Kipperman, Morristown, NJ (US)

(21) Appl. No.: 16/207,555

(22) Filed: Dec. 3, 2018

Related U.S. Application Data
(60) Provisional application No. 62/594,152, filed on Dec. 4, 2017.

(51) Int. Cl. A61F 2/06; A61M 27/00

(52) U.S. Cl. CPC A61F 2/06; A61F 2002/068; A61M 27/002

(57) ABSTRACT

The present invention comprises devices, systems and methods for providing flow modifier(s) comprising one-way, two-way or flow restrictors in one or more pulmonary veins to modify the blood pressure in a patient's left atrium or left ventricle.

### CROSS-REFERENCE TO RELATED APPLICATIONS

This application claims the benefit of U.S. Provisional Patent Application Ser. No. 62/594,152, filed Dec. 4, 2017 and entitled DEVICES AND METHOD FOR MODIFYING BLOOD PRESSURE IN THE LUNGS AND PULMONARY VASCULATURE BY IMPLANTING FLOW MODIFIER(S) IN PULMONARY VEIN(S), the entirety of which is hereby incorporated by reference.

### STATEMENT REGARDING FEDERALLY SPONSORED RESEARCH OR DEVELOPMENT

Not Applicable

### FIELD OF THE INVENTION

The invention relates to devices and methods for implanting a flow modifier comprising one or more of the group consisting of: a one-way valve, a two-way valve, and a flow restrictor in at least one pulmonary vein.

### DESCRIPTION OF THE RELATED ART

The human heart has four main chambers, the right and left ventricles, and the right and left atria. Deoxygenated blood is received by the right atrium and transmitted to the right ventricle whereby the blood is pumped through the pulmonary artery into the lungs, where the blood is oxygenated. The oxygenated blood then returns to the heart from the lungs through the pulmonary veins 100, 100′ into the left atrium. After passing through the mitral valve and into the left ventricle, the blood is pumped out of the left ventricle and into the aorta and further into the bodily arteries. During diastole, the heart relaxes and blood fills the atria and ventricles. During systole, the right and left ventricles contract and pump the blood from the right ventricle into the pulmonary artery and simultaneously from the left ventricle into the aorta. See FIG. 1.

Certain medical conditions such as pulmonary edema and pulmonary hypertension result from an increase in pressure in the lungs and/or pulmonary vasculature.

Some of these patients develop an accumulation of fluids in the lungs or pulmonary edema. Pulmonary edema may result from either failure of the left ventricle to remove blood adequately from the pulmonary circulation or an injury to the lung parenchyma or vasculature of the lung. Known treatment of pulmonary edema focuses on three strategies: improving respiratory function, treating the underlying cause, and avoiding further damage to the lung.

Further, in certain patients, blood pressure is increased pressure in the pulmonary arteries, resulting in a condition known as pulmonary hypertension. Generally, pulmonary hypertension begins with inflammation and changes in the cells that line the pulmonary arteries. Other factors may also cause the development of pulmonary hypertension, such as, e.g.: the pulmonary artery wall tighten or become less compliant than normal; the pulmonary artery walls may also be stiff at birth or become stiff from an overgrowth of cells thereon; and blood clots formation in the pulmonary arteries. Each of these conditions or factors will make it difficult for the right ventricle to push blood to the lungs via the pulmonary arteries. As a result, the pressure in the pulmonary arteries rises and the right ventricle becomes strained and weak over time. Ultimately the right ventricle may become so weakened that it cannot pump enough blood to the lungs, resulting in heart failure which is the most common cause of death in patients with pulmonary hypertension.

The present invention addresses, inter alia, these problems.

### BRIEF DESCRIPTION OF THE SEVERAL VIEWS OF THE DRAWINGS

FIG. 1 illustrates the basic blood flow of the human heart.

FIG. 2 illustrates a cutaway cross-sectional view of one embodiment of the present invention.

FIG. 3 illustrates a cutaway cross-sectional view of one embodiment of the present invention.

FIG. 4 illustrates a cutaway cross-sectional view of one embodiment of the present invention.

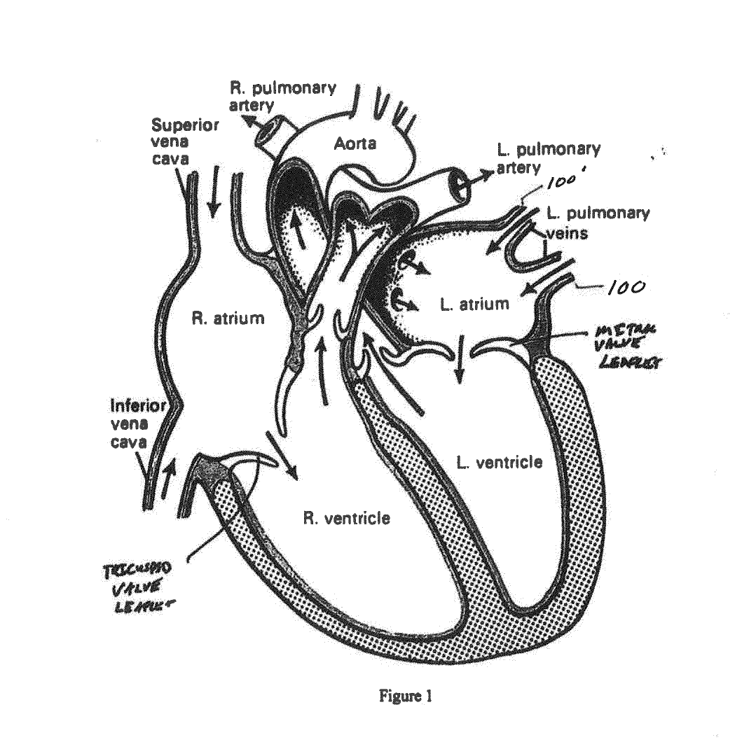

### BRIEF SUMMARY OF THE INVENTION

The present invention comprises devices, systems and methods for providing flow modifier(s) comprising one-way, two-way or flow restrictors in one or more pulmonary veins to modify the blood pressure in a patient's left atrium or left ventricle.

### DETAILED DESCRIPTION OF THE INVENTION

Generally, the various embodiments of the present invention comprise devices and methods for reducing the pressure in a patient's lungs and/or pulmonary vasculature.

More specifically, as shown in FIG. 2, a device and method comprise inserting and implanting a flow modifier 200 within one or more of a patient's pulmonary veins 100, 100′. The flow modifier 200 may comprise a one-way valve, a two-way valve, and/or a flow restrictor. A one-way valve within a pulmonary vein may allow one-way blood flow from the lungs to the left atrium but backflow from the left atrium to the lungs is not permitted. A two-way valve may allow bidirectional flow from the lungs to the left atrium, but may function to limit flow in one direction more than the other direction, e.g., more blood flow from the lungs to the left atrium is permitted compared with the backflow from the left atrium to the lungs. A flow restrictor may function to restrict the flow of blood within the pulmonary vein(s) by either reducing the effective diameter of the pulmonary vein lumen, reducing the effective surface area of the pulmonary lumen through use of a screen or other similar mechanism, and/or by providing a material within the flow restrictor that slows and/or limits the flow of blood therethrough. The flow restrictor may comprise a one-way restriction on blood flow, leaving the opposite flow unchanged or unrestricted. Alternatively, the flow restrictor may comprise a two-way restriction on blood flow, either with a substantially equivalent restriction in bidirectional blood flow or with a proportionally greater restriction of blood flow in one direction, e.g., the flow from the lungs to the left atrium, than in the other direction. Known flow restrictors are disclosed in the following: U.S. Pat. Nos. 4,456,014; 4,560,375; 6,086,527; and 6,254,564, the contents of each of which are hereby incorporated in their entirety.

As will now be apparent to the skilled artisan, the blood flow volume on either side, i.e., the upstream side and the downstream side of the flow modifier 200 within the pulmonary vein 100, 100′, may be modified using one or more flow modifiers 200 as described herein.

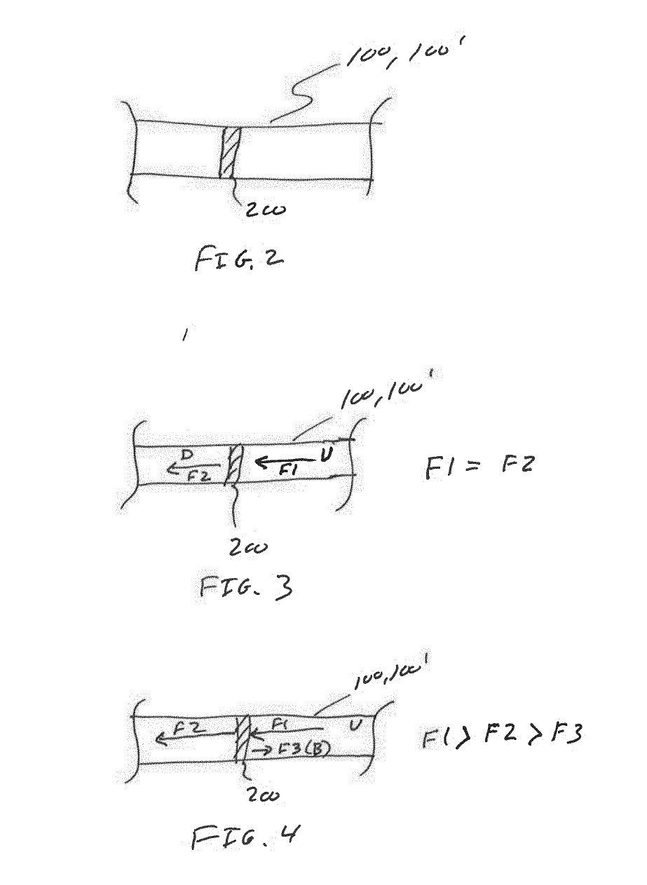

FIG. 3 shows the flow modifier 200 that allows no backflow in the upstream direction flow U across the flow modifier 200 from the downstream fluid flow D. Thus, upstream flow volume F1 is equal to downstream flow volume F2. In this case, the flow modifier 200 may comprise a one-way valve or a flow restrictor that functions as a one-way valve, wherein backflow is not allowed and fluid flow is allowed in only one direction.

FIG. 4 provides a flow modifier 200 that may allow some backflow through modifier 200 within the pulmonary vein 100, 100′. In this case, as shown, upstream U flow F1 may comprise a greater flow volume that downstream D flow F2 as a result of a regulated volume of backflow B shown as F3 through flow modifier 200.

The flow modifier device 200 comprising a one-way valve, a two-way valve and/or a flow restrictor may be provided or delivered to the pulmonary vein(s) in operable engagement and combination with an expandable stent 300, expandable stents 300 being well known in the art, wherein the flow modifier device 200 is attached to, or may be integrated within, the interior I of the expandable stent frame 300. When the stent 300 is expanded, it will engage the pulmonary vein 100, 100′ inner walls and engage the walls. Alternatively, the flow modifier device 200 may be delivered and implanted into one or more of the pulmonary veins 100, 100′ without an expandable stent using an expandable conduit or other structure that may be implanted in the lumen of the pulmonary vein.

The number of the flow modifier devices 200, each one implanted in an individual pulmonary vein 100, 100′, required for a particular patient will vary depending on the pressure reduction needs. In addition, the pulmonary vein(s) 100, 100′ that are selected for implantation with the flow modifier(s) 200 will depend on the patient's pressure reduction needs. Further, the form or type of the flow modifier device, e.g., a one-way valve, or a two-way valve, or a flow restrictor, used within a particular selected pulmonary vein to modify and/or reduce blood flow therein may depend upon the individual patient's pressure reduction needs. Finally, the magnitude of the pressure reduction provided by the selected flow modifier within the selected pulmonary vein may be customized to meet the patient's pressure reduction needs.

The pressure of blood flow from the patient's lungs may be modified using the various embodiments of the present invention to, e.g., lower the pressure within the patient's left atrium and, in turn, the patient's left ventricle by restricting the flow and pressure within one or more pulmonary veins.

Accordingly, conditions such as pulmonary edema and pulmonary hypertension may be treated using the various embodiments of the device by implanting one or more flow modifier devices 200 in at least one of the patient's pulmonary veins.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A flow modifier disposed within at least one pulmonary vein adapted to regulate fluid flow through the at least one pulmonary vein.

2. The flow modifier of claim 1, further comprising a one-way valve.

3. The flow modifier of claim 1, further comprising a two-way valve.

4. The flow modifier of claim 1, further comprising a flow restrictor.

5. The flow modifier of claim 1, further comprising selecting the flow modifier from at least one of the group consisting of: a one-way valve, a two-way valve, and a flow restrictor.

6. The flow modifier of claim 1, further comprising a flow volume upstream of the flow modifier, a flow volume downstream of the flow modifier,
wherein the flow volume upstream of the flow modifier and the flow volume downstream of the flow modifier are substantially equal.

7. The flow modifier of claim 6, wherein no backflow is allowed through the flow modifier to the upstream flow volume.

8. The flow modifier of claim 1, further comprising the fluid modifier configured to allow no backflow of fluid flow through the flow modifier.

9. The flow modifier of claim 1, further comprising the fluid modifier configured to allow some backflow of fluid flow through the flow modifier.

10. A method for modifying the blood flow and pressure within a patient's left atrium and left ventricle, comprising:
providing a flow modifier;
delivering the flow modifier to at least one pulmonary vein;
implanting the flow modifier within the at least one pulmonary vein; and
modifying the blood flow and pressure within the patient's left atrium and left ventricle.

11. The method of claim 10, further comprising providing, delivering and implanting more than one flow modifier to one or more of the patient's pulmonary veins.

12. The method of claim 10, further comprising modifying the blood flow and pressure within the patient's lungs and pulmonary vasculature.

13. A method for treating pulmonary edema in a patient, comprising:
providing a flow modifier;
delivering the flow modifier to at least one pulmonary vein;
implanting the flow modifier within the at least one pulmonary vein;
modifying the blood flow and pressure within the patient's lungs; and
treating the patient's pulmonary edema.

14. The method of claim 13, further comprising providing, delivering and implanting more than one flow modifier to one or more of the patient's pulmonary veins.

15. A method for treating pulmonary hypertension in a patient, comprising:
providing a flow modifier;
delivering the flow modifier to at least one pulmonary vein;
implanting the flow modifier within the at least one pulmonary vein;
modifying the blood flow and pressure within the patient's lungs and
treating the patient's pulmonary hypertension.

16. The method of claim 15, further comprising providing, delivering and implanting more than one flow modifier to one or more of the patient's pulmonary veins.
